(19) United States
(12) Patent Application Publication
(10) Pub. No.: US 2009/0093843 A1
(43) Pub. Date: Apr. 9, 2009

(54) DYNAMIC SPINE STABILIZATION SYSTEM

(76) Inventors: Jeremy J. Lemoine, Austin, TX (US); Wayne Gray, Pflugerville, TX (US); Kameron Scott Ely, Cedar Park, TX (US); Marc M. Peterman, Austin, TX (US)

Correspondence Address: SPRINKLE IP LAW GROUP, 1301 W. 25TH STREET, SUITE 408, AUSTIN, TX, 78705, US

(21) Appl. No.: 11/868,183

(22) Filed: Oct. 5, 2007

(51) Int. Cl. A61B 17/58; A61B 17/08; A61B 17/70; A61B 17/04

(52) U.S. Cl. 606/246; 606/103; 606/151; 606/324; 606/330; 606/326; 606/264; 606/301; 606/308; 606/278

(57) ABSTRACT

Disclosed are devices and systems for spinal fixation and dynamic stabilization. Elongated members may be positioned lateral to spinous processes and coupled to two or more vertebrae. Interspinous members may be positioned between adjacent spinous processes and coupled to the elongated members. One embodiment of an interspinous member comprises a body and two lateral portions. The lateral portions may couple to the elongated members to position the interspinous members. An interspinous member and the positioning of the interspinous member may allow selected movement between the adjacent spinous processes.

### TECHNICAL FIELD

This disclosure relates generally to spinal implants and spine stabilization systems, and more particularly to apparatus and systems for stabilizing movement between spinous processes.

### BACKGROUND OF THE DISCLOSURE

The human spine consists of segments known as vertebrae linked by intervertebral disks and held together by ligaments. There are 24 movable vertebrae—7 cervical, 12 thoracic, and 5 lumbar. Each vertebra has a somewhat cylindrical bony body (centrum), a number of winglike projections, and a bony arch. The bodies of the vertebrae form the supporting column of the skeleton. The arches are positioned so that the space they enclose forms the vertebral canal. It houses and protects the spinal cord, and within it the spinal fluid circulates. Ligaments and muscles are attached to various projections of the vertebrae.

The spine is subject to abnormal curvature, injury, infections, tumor formation, arthritic disorders, and puncture or slippage of the cartilage disks. Injury or illness, such as spinal stenosis and prolapsed discs may result in intervertebral discs having a reduced disc height, which may lead to pain, loss of functionality, reduced range of motion, and the like. Scoliosis is one relatively common disease which affects the spinal column. It involves moderate to severe lateral curvature of the spine, and, if not treated, may lead to serious deformities later in life. One treatment involves surgically implanting devices to correct the curvature.

Modern spine surgery often involves spinal fixation through the use of spinal implants or fixation systems to correct or treat various spine disorders or to support the spine. Spinal implants may help, for example, to stabilize the spine, correct deformities of the spine, facilitate fusion, or treat spinal fractures.

A spinal fixation system typically includes corrective spinal instrumentation that is attached to selected vertebra of the spine by screws, hooks, and clamps. The corrective spinal instrumentation includes spinal rods or plates that are generally parallel to the patient's back. The corrective spinal instrumentation may also include transverse connecting rods that extend between neighboring spinal rods. Spinal fixation systems are used to correct problems in the cervical, thoracic, and lumbar portions of the spine, and are often installed posterior to the spine on opposite sides of the spinous process and adjacent to the transverse process.

Often, spinal fixation may include rigid (i.e., in a fusion procedure) support for the affected regions of the spine. Such systems limit movement in the affected regions in virtually all directions (e.g., in a fused region). More recently, so called “dynamic” systems have been introduced wherein the implants allow at least some movement (e.g., flexion, extension, lateral bending, or torsional rotation) of the affected regions in at least some directions.

### SUMMARY OF THE DISCLOSURE

Embodiments of the present disclosure offer devices and systems for stabilizing portions of the spine. Furthermore, embodiments of the present disclosure may provide spinal stabilization utilizing the spinous processes. One embodiment may be directed to an interspinous member for stabilizing movement between adjacent vertebrae, including a body for stabilizing movement between a first spinous process and a second spinous process and two lateral portions extending from the body for engaging the two elongated members positioned lateral to the first spinous process and the second spinous process to stabilize movement between the elongated members. In some embodiments, the body may have a first surface for contact with an inferior portion of the first spinous process and a second surface for simultaneous contact with a superior portion of the second spinous process. In some embodiments, the upper surface has a curved portion for contact with a portion of the inferior portion of a spinous process. In some embodiments, the lower surface has a curved portion for contact with a portion of the superior portion of a spinous process. In some embodiments, each lateral portion is curved to receive a portion of an elongated member such that the lateral portion engages the elongated member. In some embodiments, each lateral portion comprises a receiving portion for receiving a portion of an elongated member.

Another embodiment of the present disclosure is directed to a system for stabilizing movement between two or more vertebrae, including two elongated members for positioning lateral to two spinous processes of the vertebrae, two fasteners for coupling each elongated member to the vertebrae, and an interspinous member for coupling to the two elongated members for positioning between the spinal processes of adjacent spinous processes. In some embodiments, each interspinous member includes a body for stabilizing movement between a first spinous process and a second spinous process, having a first surface for contact with an inferior portion of the first spinous process and a second surface for simultaneous contact with a superior portion of the second spinous process, and two lateral portions extending from the body for engaging the two elongated members positioned lateral to the first spinous process and the second spinous process to stabilize movement between the elongated members. In some embodiments, positioning the lateral portions on the elongated members positions the body to stabilize movement between adjacent spinous processes. In some embodiments, a fastener comprises a bone fastener assembly. In some embodiments, a fastener comprises a hook. In some embodiments, a fastener comprises a bolt for passage through a portion of a spinous process. In some embodiments, a fastener comprises a clamp. In some embodiments, a fastener comprises a universal band. In some embodiments, the two elongated members are connected at both ends and comprise a slot along a portion thereof to accommodate two spinous processes. In some embodiments, a slot formed along a portion of a single rod divides the rod into two elongated members for positioning lateral to two spinous processes. In some embodiments, the two elongated members comprise an engagement feature, wherein one or more interspinous members engage the elongated members at one of the engagement features.

One embodiment of the disclosure is directed to method for stabilizing a portion of a spine, including coupling a first elongated member to a first side of a first vertebra, coupling the first elongated member to the first side of a second vertebra, coupling a second elongated member to a second side of the first vertebra, coupling the second elongated member to the second side of the second vertebra, positioning a body of an interspinous member between adjacent spinous processes such that a first surface of the body contacts an inferior portion of a first spinous process and a second surface contacts a superior portion of a second spinous process, and engaging two lateral portions of the interspinous member to a portion of the two elongated members. In some embodiments, the first or second elongated member spans between the first and second vertebrae in some embodiments, the body stabilizes movement between the adjacent spinous processes. In some embodiments, each lateral portion receives a portion of an elongated member. In some embodiments, the positioning of the lateral portions on the elongated members positions the body to stabilize movement between adjacent spinous processes. In some embodiments, a lateral portion is slidably positioned along the first or second elongated member. In some embodiments, the method includes engaging the interspinous member with an engagement feature on an elongated member. In some embodiments, the method may include coupling an elongated member to a bone fastener assembly or hook implantable in bony tissue. In some embodiments, the method may include coupling an elongated member to a portion of a bolt assembly for penetrating through a portion of a spinous process. In some embodiments, the method may include coupling one of the elongated members to the other elongated member. In some embodiments, the method may include coupling one of the elongated members to the other elongated member with a universal band.

One advantage to implanting this type of system is the decreased number of bone fasteners, which may result in less time in surgery, decreased risk of damage to the spinal cord, spinal column, muscles, blood vessels, nerves, or other tissues or organs, and/or reduced recovery time, decreased complications such as pain or infection, or other surgical or subsequent issues.

These, and other, aspects of the disclosure will be better appreciated and understood when considered in conjunction with the following description and the accompanying drawings. The following description, while indicating various embodiments of the disclosure and numerous specific details thereof, is given by way of illustration and not of limitation. Many substitutions, modifications, additions or rearrangements may be made within the scope of the disclosure, and the disclosure includes all such substitutions, modifications, additions or rearrangements.

### BRIEF DESCRIPTION OF THE DRAWINGS

FIG. 1 depicts a perspective view of one embodiment of a spinal stabilization system.

FIG. 2 depicts a perspective view of one embodiment of a bone fastener assembly.

FIG. 3 depicts a cross-sectional view of one embodiment of a bone fastener assembly.

FIG. 4 depicts a perspective view of one embodiment of a bone fastener assembly.

FIGS. 5A and 5B depict superior and posterior views of a vertebral body having one embodiment of a spinal stabilization system implanted thereon, the spinal stabilization system having adjustable bone fastener assemblies.

FIG. 6 depicts one embodiment of an elongated member.

FIG. 7 depicts one embodiment of an elongated member.

FIG. 8 depicts one embodiment of an elongated member.

FIG. 9 depicts one embodiment of an elongated member.

FIG. 10 depicts an exploded cross-sectional view of one embodiment of a hook.

FIG. 11 depicts a cross-sectional view of one embodiment of a hook.

FIG. 12 depicts an exploded perspective view of one embodiment of a hook.

FIG. 13 depicts a perspective view of one embodiment of a hook.

FIG. 14 depicts an exploded view of one embodiment of a clamp.

FIG. 15 depicts a perspective view of one embodiment of a clamp attached to a portion of a spine.

FIG. 16 depicts an exploded view of one embodiment of a clamp.

FIG. 17 depicts a perspective view of one embodiment of a clamp.

FIG. 18 depicts a perspective view of one embodiment of a universal band.

FIG. 19 depicts a cross-sectional view of one embodiment of a universal band.

FIG. 20 depicts a cutaway view of one embodiment of a universal band attached to a portion of a spine.

FIG. 21 depicts a perspective view of one embodiment of a universal band attached to a portion of a spine.

FIG. 22 depicts a perspective view of an exemplary spinal stabilization system.

FIG. 23 depicts a posterior view of one embodiment of a spinal stabilization system.

FIG. 24 depicts a sagittal view of one embodiment of a dynamic stabilization system.

FIG. 25 depicts a cross-sectional view of one embodiment of an interspinous member.

FIG. 26 depicts a perspective view of one embodiment of an interspinous member.

FIG. 27 depicts a perspective view of a portion of one embodiment of a spinal stabilization system.

FIG. 28 depicts a perspective view of a portion of one embodiment of a spinal stabilization system.

FIG. 29 depicts a cross-section sectional view of a portion of one embodiment of a spinal stabilization system.

### DETAILED DESCRIPTION OF PREFERRED EMBODIMENTS

The disclosure and the various features and advantageous details thereof are explained more fully with reference to the non-limiting embodiments that are illustrated in the accompanying drawings and detailed in the following description. Descriptions of well known starting materials, processing techniques, components and equipment are omitted so as not to unnecessarily obscure the disclosure in detail. Skilled artisans should understand, however, that the detailed description and the specific examples, while disclosing preferred embodiments of the disclosure, are given by way of illustration only and not by way of limitation. Various substitutions, modifications, additions or rearrangements within the scope of the underlying inventive concept(s) will become apparent to those skilled in the art after reading this disclosure.

A spinal stabilization system may be installed in a patient to stabilize a portion of a spine. Spinal stabilization may be used, but is not limited to use, in patients having degenerative disc disease, spinal stenosis, spondylolisthesis, pseudoarthrosis, and/or spinal deformities; in patients having fracture or other vertebral trauma; and in patients after tumor resection. A spinal stabilization system may be installed using a minimally invasive procedure. An instrumentation set may include instruments and spinal stabilization system components for forming a spinal stabilization system in a patient.

A minimally invasive procedure may be used to limit an amount of trauma to soft tissue surrounding vertebrae that are to be stabilized. In some embodiments, the natural flexibility of skin and soft tissue may be used to limit the length and/or depth of an incision or incisions needed during the stabilization procedure. Minimally invasive procedures may provide limited direct visibility in vivo. Forming a spinal stabilization system using a minimally invasive procedure may include using tools to position system components in the body.

A minimally invasive procedure may be performed after installation of one or more spinal implants in a patient. The spinal implant or spinal implants may be inserted using an anterior procedure, a posterior procedure, a lateral procedure, or any combination thereof. The patient may be turned and a minimally invasive procedure may be used to install a posterior spinal stabilization system. A minimally invasive procedure for stabilizing the spine may be performed without prior insertion of one or more spinal implants in some patients. In some patients, a minimally invasive procedure may be used to install a spinal stabilization system after one or more spinal implants are inserted using a posterior spinal approach.

A spinal stabilization system may be used to achieve rigid pedicle fixation while minimizing the amount of damage to surrounding tissue. In some embodiments, a dynamic spinous process stabilization system may be used to provide stability to two adjacent vertebrae (i.e., one vertebral level).

In one embodiment, a dynamic spinous process stabilization system may be coupled to one or more vertebrae using one or more fasteners. An elongated member may be coupled to the fastener. As used herein, “coupled” components may directly contact each other or may be separated by one or more intervening members.

In one embodiment, a fastener may include a bone fastener assembly. A bone fastener assembly may be positioned in one of the vertebrae to be stabilized. In one embodiment, a fastener may include a hook. A hook may be positioned in one of the vertebrae to be stabilized. An elongated member may be coupled to the hook.

In one embodiment, a fastener may include a bolt. A bolt may be passed through the spinous process of one of the vertebrae to be stabilized. One end of an elongated member may be coupled and secured to the bolt on one side of the spinous process and one end of the other elongated member may be coupled to the bolt on the other side of the spinous process.

In one embodiment, a fastener may include a universal band. In one embodiment, a flexible ligature of a universal band may be looped around a portion of one of the vertebrae to be stabilized and pass through a portion of a universal band body. In one embodiment, an elongated member may be coupled to a collar coupled to portion of the universal band body. In one embodiment, the flexible ligature may be tightened.

In one embodiment, a fastener may include a clamp. In one embodiment, a clamp may be positioned about one of the vertebrae to be stabilized. In one embodiment, an elongated member may be coupled to the clamp and the clamp may be tightened to couple the elongated member to the vertebrae. Alternatively, the ends of both elongated members may be coupled to the clamp and the clamp may be tightened to couple the elongated members to the vertebrae.

In some embodiments, a dynamic stabilization system may be installed between two vertebrae (i.e., a single vertebral level) in a patient. Such a system may be referred to as a single-level stabilization system. In some embodiments, a spinal stabilization system may provide stability to three or more vertebrae (i.e., two or more vertebral levels). A dynamic stabilization system may include one or more fasteners such as bone fastener assemblies, hooks, bolts, universal bands (u-bands), and/or clamps. One or more fasteners may be positioned on each end vertebra of the vertebrae to be stabilized. In one embodiment an elongated member may be coupled to the fasteners.

Embodiments of the dynamic spinal stabilization system disclosed herein are particularly suited for minimally invasive procedures which have many desirable advantages. For example, minimally invasive procedures may reduce trauma to soft tissue surrounding vertebrae that are to be stabilized. In a minimally invasive surgery, only a small opening may need to be made in a patient. As an example, for a single-level stabilization procedure, the surgical procedure may be performed through 2 cm to 4 cm incisions formed in the skin of the patient. In some embodiments, the incision may be above and substantially between the vertebrae to be stabilized. In some embodiments, the incision may be above and between the vertebrae to be stabilized. In some embodiments, the incision may be above and substantially halfway between the vertebrae to be stabilized. A minimally invasive procedure may reduce an amount of post-operative pain felt by a patient as compared to invasive spinal stabilization procedures. A minimally invasive procedure may reduce recovery time for the patient as compared to invasive spinal procedures.

Spinal stabilization systems may be used to correct problems in lumbar, thoracic, and/or cervical portions of a spine. Various embodiments of a spinal stabilization system may be used from the C1 vertebra to the sacrum. For example, a spinal stabilization system may be implanted posterior to the spine to maintain distraction between adjacent vertebral bodies in a lumbar portion of the spine.

Components of spinal stabilization systems may be made of materials including, but not limited to, titanium, titanium alloys, stainless steel, ceramics, and/or polymers. Some components of a spinal stabilization system may be autoclaved and/or chemically sterilized. Components that may not be autoclaved and/or chemically sterilized may be made of sterile materials. Components made of sterile materials may be placed in working relation to other sterile components during assembly of a spinal stabilization system.

Some embodiments of dynamic stabilization systems disclosed herein may provide greater freedom of movement to facilitate the healing process. In some embodiments, dynamic stabilization systems allow for motion such as twisting, lateral bending, torsion, flexion, and extension. In some embodiments, a dynamic stabilization system may be coupled to one or more spinous processes, transverse processes, and/or pedicles.

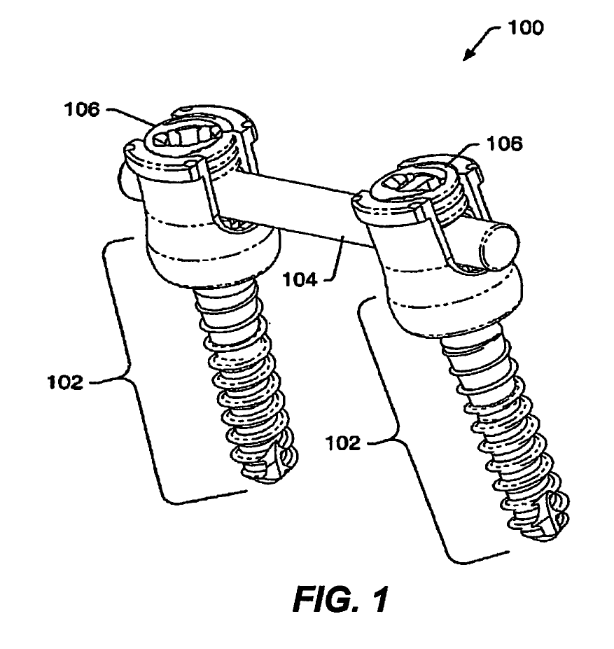

FIG. 1 depicts a portion of one embodiment of spinal stabilization system 100. In FIG. 1, a portion (i.e., one side) of spine stabilization system 100 may have elongated member 104 coupled to two or more bone fastener assemblies 102, which can be positioned lateral to the spinous processes and coupled to the vertebrae. Spinal stabilization system 100 may include two elongated members 104 coupled to two or more bone fastener assemblies 102 on two or more vertebrae such that elongated members 104 span a portion of the spine. Other spinal stabilization system embodiments may include, but are not limited to, plates and fasteners including bone fastener assemblies 102, hooks, bolts, clamps, and universal bands. In some embodiments, spinal stabilization system 100 may span three or more vertebrae and used as a multi-level spinal stabilization system. In some embodiments, a multi-level spinal stabilization system may include additional bone fastener assemblies 102, hooks, bolts, clamps, and universal bands to couple to one or more other vertebrae.

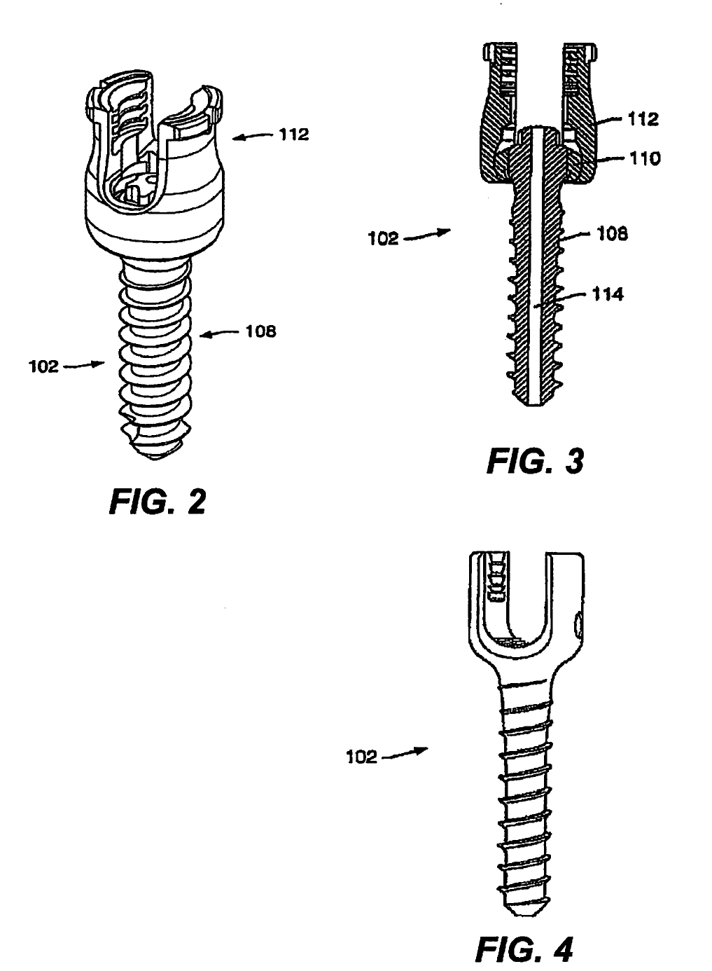

FIG. 2 depicts a perspective view of bone fastener assembly 102. Bone fastener 108 may be, but is not limited to, a bone screw, a ring shank fastener, a barb, a nail, a brad, or a trocar. Bone fasteners 108 and/or bone fastener assemblies 102 may be provided in various lengths in an instrumentation set to accommodate variability in vertebrae. For example, an instrumentation set for stabilizing vertebrae in a lumbar region of the spine may include bone fastener assemblies 102 with lengths ranging from about 30 mm to about 75 mm in 5 mm increments. Bone fastener assembly 102 may be stamped with indicia (i.e., printing on a side of the collar). In some embodiments, bone fastener assembly 102 or bone fastener 108 may be color-coded to indicate a length of the bone fastener 108. In some embodiments, bone fastener 108 with a 30 mm thread length may have a magenta color, bone fastener 108 with a 35 mm thread length may have an orange color, and bone fastener 108 with a 55 mm thread length may have a blue color. Other colors may be used as desired.

Each bone fastener 108 provided in an instrumentation set may have substantially the same thread profile and thread pitch. In one embodiment, the thread may have about a 4 mm major diameter and about a 2.5 mm minor diameter with a cancellous thread profile. In some embodiments, the minor diameter of the thread may be in a range from about 1.5 mm to about 4 mm or larger. In some embodiments, the major diameter of the thread may be in a range from about 3.5 mm to about 6.5 mm or larger. Bone fasteners 108 with other thread dimensions and/or thread profiles may also be used. A thread profile of bone fasteners 108 may allow bone purchase to be maximized when bone fastener 108 is positioned in vertebral bone.

As used herein, the term “collar” includes any element that wholly or partially encloses or receives one or more other elements. Collar 112 may enclose or receive elements including, but not limited to, bone fastener 108, closure member 106, ring 110, and/or elongated member 104. In some embodiments, collar 112 may couple two or more other elements together (e.g., elongated member 104 and bone fastener 108). Collar 112 may have any of various physical forms. In some embodiments, collar 112 may have a “U” shape, however it is to be understood that collar 112 may also have other shapes.

In some embodiments, collar 112 may be open or closed. A collar having a slot and an open top, such as collar 112 shown in FIG. 2 may be referred to as an “open collar.” A fastener that includes an open collar 112 may be referred to as an “open fastener.” In some embodiments, elongated member 104 may be top loaded into the open fastener. A closure member may be coupled to collar 112 to secure elongated member 104 to the open fastener. A collar that does not include a slot and an open top may be referred to as a “closed collar.” A spinal implant that includes a closed collar may be referred to as a “closed implant.” In some embodiments, a closed collar may include an aperture, bore, or other feature in side surfaces for accommodating other components of a stabilization system (e.g., an elongated member). A connection member may be used to couple elongated member 104 to a closed implant.

In some embodiments, closure member 106 may be coupled to collar 112 of bone fastener assembly 102 to couple elongated member 104 positioned in collar 112 to bone fastener assembly 102. In some embodiments, closure member 106 may be cannulated. In some embodiments, closure member 106 may have a solid central core. In some embodiments, closure member 106 with a solid central core may provide a more secure connection to elongated member 104 than a cannulated closure member 106 by providing contact against elongated member 104 at a central portion of closure member 106 as well as near an edge of closure member 106.

FIG. 3 depicts a cross-sectional representation of bone fastener 108, ring 110, and collar 112 of bone fastener assembly 102. Bone fastener 108 of bone fastener assembly 102 may include passage 114. Bone fastener 108 may be cannulated (i.e., passage 114 may run through the full length of bone fastener 108). In one embodiment, a guide wire may be placed through passage 114 so that bone fastener 108 may be inserted into a vertebra at a desired location and in a desired angular orientation relative to the vertebra with limited or no visibility of the vertebra.

In some embodiments, bone fastener assembly 102 may be a fixed angle fastener. FIG. 4 depicts one embodiment of bone fastener 108 having a fixed angle. Collar 112 and bone fastener 108 may be formed as a unitary piece of material (e.g., stainless steel, titanium, titanium alloys, ceramics, and/or polymers). A fixed angle fastener may be positioned as the first bone fastener assembly inserted into a vertebra.

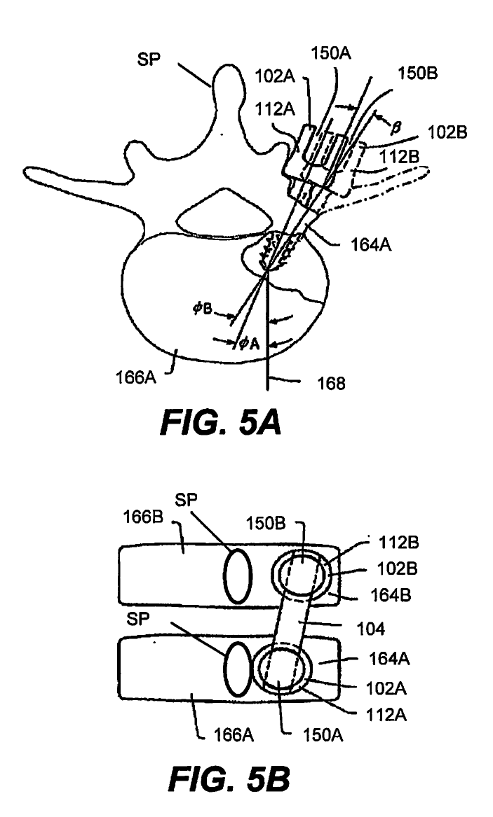

In some embodiments, one or more biased collars 112 may be used in a spinal stabilization system. In some embodiments, the spinal stabilization systems may be a multi-level system. In some embodiments, biased collars 112 may be used to accommodate the increasing angle of the pedicle corridor for each lumbar vertebra. In some embodiments, the angle may increase by about 5 degrees for each successive lumbar vertebra. FIGS. 5A and 5B depict superior and posterior views of one embodiment of a spinal stabilization system including bone fastener assembly 102A coupled to pedicle 164A and vertebra 166A and bone fastener assembly 102B coupled to pedicle 164B and vertebra 166B.

In some embodiments, bone fastener 108 of bone fastener assembly 102A may engage pedicle 164A at pedicle angle φA (phi-Alpha) relative to sagittal plane 168. Pedicle angle φA (phi-Alpha) may range between about 13 degrees and about 17 degrees. In some embodiments, collar 112A of bone fastener assembly 102A may be unbiased. Pedicle angle φβ (phi-Beta) may range between about 18 degrees and about 22 degrees. In some embodiments, collar 112B may have a bias angle β (Beta) of about 5 degrees. In some embodiments, bone fastener assembly 102B may engage pedicle 164B at pedicle angle φβ (phi-Beta). Because the bias of collar 112B is approximately equal to the difference between the pedicle angles of the two vertebrae, slots 150A and 150B in bone fastener assemblies 102A and 102B, respectively, may be generally aligned when both bone fasteners 108 are in neutral positions.

In some embodiments, angulation of either or both collars 112 of the bone fastener assemblies 102 may allow fine adjustment of engagement angles of the bone fasteners 108. In some embodiments, angulation of collars 112 may allow adjustment in the orientation of bone fasteners 108 in a sagittal plane (i.e., to conform to lordosis of a spine) while still allowing collars 112 to be easily coupled with elongated member 104. In some embodiments, elongated member 104 may be disposed in slots 150A and 150B and coupled to collars 112 by closure members. In some embodiments, a flexible driver or a polyaxial driver (e.g., a driver with a universal joint) may be used to drive the heads of bone fasteners 108 from a position that is off-axis from bone fasteners 108 to reduce the size of an opening of the body needed to implant the spinal stabilization system.

In some embodiments, various instruments may be used in minimally invasive spine stabilization procedures to form a spinal stabilization system in a patient. The instruments may include, but are not limited to, positioning needles, guide wires, dilators, bone awls, bone taps, sleeves, drivers, tissue wedges, elongated member length estimating tools, mallets, tissue retractors, tensioning tools, and tissue dilators. In some embodiments, the instruments may be provided in an instrumentation set. In some embodiments, the instrumentation set may also include components of the spinal stabilization system.

In some embodiments, instruments used to install a spinal stabilization system may be made of materials including, but not limited to, stainless steel, titanium, titanium alloys, ceramics, and/or polymers. Some instruments may be autoclaved and/or chemically sterilized. Some instruments may include components that cannot be autoclaved or chemically sterilized. Components of instruments that cannot be autoclaved or chemically sterilized may be made of sterile materials. The sterile materials may be placed in working relation to other parts of the instrument that have been sterilized.

In some embodiments, elongated member 104 may be cut to length and contoured as desired. In some embodiments, a medical practitioner may use experience and judgment to determine curvature of elongated member 104 for a patient. In some embodiments, a desired curvature for elongated member 104 may be determined using fluoroscopic imaging.

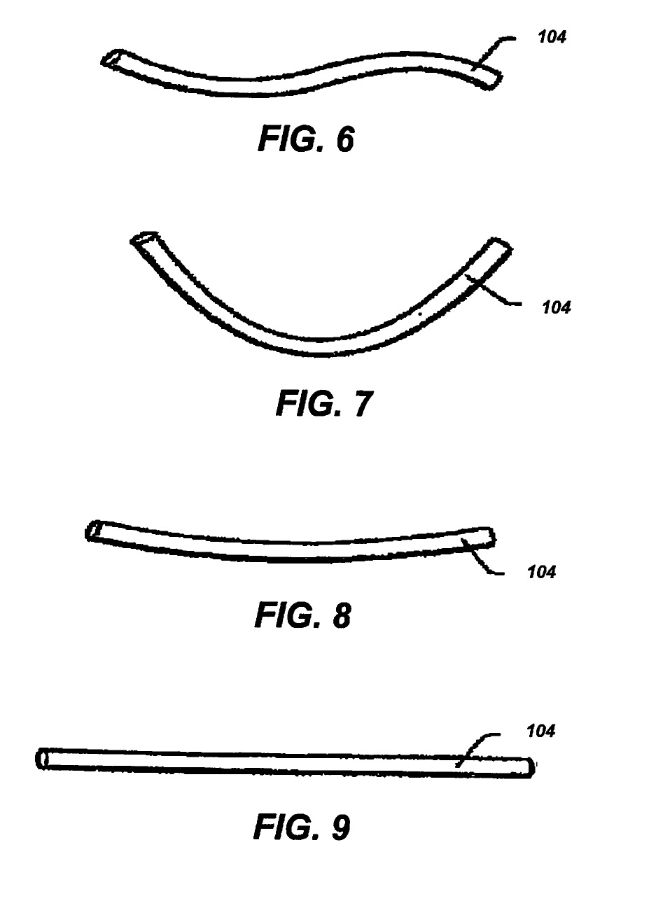

In some embodiments, elongated members 104 may have shapes including, but not limited to, straight, bent, curved, s-shaped, and z-shaped. FIG. 6 depicts one embodiment of S-shaped elongated member 104. FIG. 7 depicts one embodiment of angled elongated member 104. FIG. 8 depicts one embodiment of bent elongated member 104. FIG. 9 depicts one embodiment of straight elongated member 104. In some embodiments, elongated members 104 may have a substantially circular longitudinal cross section. In some embodiments, elongated members 104 may have other cross-sectional shapes including, but not limited to, regular shapes (oval, rectangular, rhomboidal, square) and irregular shapes. An instrumentation kit for a spinal stabilization system may include straight elongated members 104 and/or pre-shaped elongated members 104. Straight elongated members 104 and/or pre-shaped elongated members 104 may be contoured to accommodate patient anatomy if needed during the surgical procedure. In some embodiments, elongated members 104 may include notches, indentations, alignment marks, protrusions, holes, or other engagement features for engagement by an interspinous member. In some embodiments, an engagement feature may provide a surgeon with a reference point to help visualize the placement or orientation of interspinous member or elongated member 104. In some embodiments, an engagement feature may enable the surgeon to couple the interspinous member to a particular position on elongated member 104. For example, a hole or recessed portion on elongated member 104 may receive a protrusion from interspinous member to securely couple interspinous member to elongated member.

In some embodiments, insertion of elongated member 104 may not be visualized subcutaneously. In some embodiments, a positioning tool may be used to guide elongated member 104 into collars 112. In some embodiments, with slight pressure, elongated member 104 may be rotated subcutaneously into a substantially horizontal position and seated in collars 112.

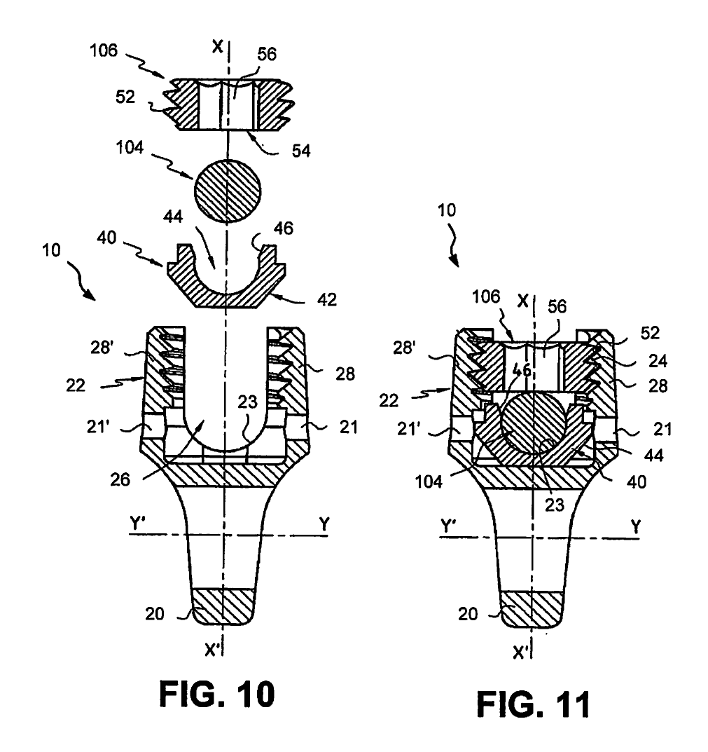

In some embodiments, spinal stabilization system 100 may include hook 10. FIG. 10 depicts an exploded cross-sectional view of one embodiment of hook 10 useful for coupling elongated members 104 to vertebrae. In one embodiment, hook 10 may have curved portion 20. Curved portion 20 may couple hook 10 to a spinous process, a transverse process, or a pedicle of a vertebra.

In some embodiments, hook 10 may include assembly head 22 suitable for securing hook 10 to elongated members 104. In some embodiments, curved portion 20 and assembly head 22 may be manufactured separately. In one embodiment, assembly head 22 may be mechanically joined to curved portion 20. In one embodiment, assembly head 22 may be threadably joined to curved portion 20. In one embodiment, assembly head 22 may be compression fit to curved portion 20. In one embodiment, assembly head 22 may be chemically joined to curved portion 20. In one embodiment, assembly head 22 may be glued to curved portion 20. In one embodiment, assembly head 22 may be epoxied to curved portion 20. In some embodiments, assembly head 22 may be thermally joined to curved portion 20. In one embodiment, assembly head 22 may be welded to curved portion 20. In one embodiment, assembly head 22 may be sweat-locked to curved portion 20.

In some embodiments, assembly head 22 and curved portion 20 may be manufactured as a single unit to form hook 10. In one embodiment, assembly head 22 and curved portion 20 may be cast. In one embodiment, assembly head 22 and curved portion 20 may be machined as a single unit.

In some embodiments, hook 10 may have transverse and longitudinal dimensions lying in the range 5 millimeters (mm) to 20 mm. In some embodiments, assembly head 22 of hook 10 may have an outer shape substantially cylindrical about the axis x-x′. In some embodiments, assembly head 22 may have an inner surface with helical thread 24 and may have slot 26, which may define two upwardly extending side walls 28, 28′.

In some embodiments, as can be seen in FIGS. 10 and 11, the section of assembly head 22 in a plane (XOY) orthogonal to the plane of symmetry of hook 10, may be substantially U-shaped, and the bottom portions of the opening may have curved profiles 23 for supporting the outside surface of elongated member 104.

In some embodiments, as can be seen in FIG. 11, the helical thread 24 of assembly head 22 may be of the “artillery” type, i.e. it may present an asymmetric trapezoidal thread. An advantage of this type of thread is that it enables the radial component of the screw-fastening force to be reduced. A further advantage of this is explained in greater detail below.

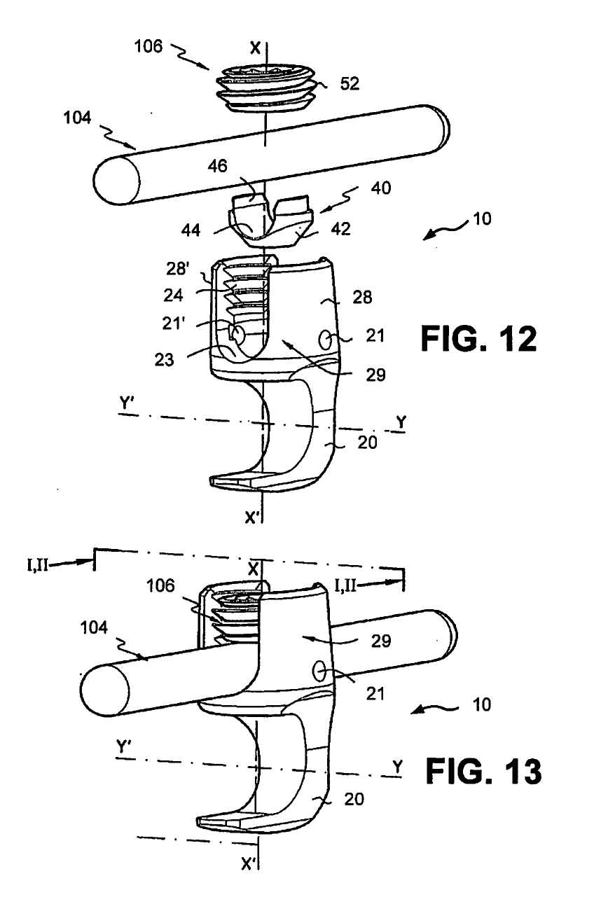

In some embodiments, assembly head 22 may include two orifices 21, 21′ situated in the side walls 28, 28′. During a surgical operation, the surgeon may use an instrument for inserting, advancing, adjusting, or otherwise positioning hook 10. In some embodiments, an instrument may have two protrusions receivable in orifices 21, 21′ in order to hold hook 10. FIG. 12 depicts an exploded perspective view of one embodiment of hook 10 for coupling elongated member 104 to a vertebra, in which orifices 21 and 21′ may be used for connection by a tool.

In some embodiments, once the surgeon has determined the type of elongated member 104, retention means 40 may be positioned in assembly head 22 to retain elongated member 104 in assembly head 22 before it is locked. An advantage is the ability for the surgeon to adjust the position of elongated member 104 and secure it without holding elongated member 104. In some embodiments, while the position of elongated member 104 is being adjusted, elongated member 104 can move in translation in retention means 40 and can also pivot about its own axis. Meanwhile, retention means 40 prevents elongated member 104 from escaping from assembly head 22.

In some embodiments, retention means 40 is in the form of clip insert 40. Several types of insert are available corresponding to elongated members 104 of different diameters. Advantageously, various inserts 40 can be fitted in the same assembly head 22.

In some embodiments, hook 10 may include closure member 106. Closure member 106 may have a form of a cylindrical part of diameter slightly smaller than the inside diameter of the opening in assembly head 22. In some embodiments, bottom portion of closure member 106 may have thread 52 to enable it to be secured to assembly head 22. In some embodiments, closure member 106 may be coupled to assembly head 22 by engaging threads 52 on closure member 106 with threads 24 in assembly head 22 and rotating closure member 106 through one-fourth of a turn.

Retaining member 40 may have an opening 44 of diameter substantially equal to the diameter of elongated member 104, and for selected contact with elongated member 104. In some embodiments, this engagement may be achieved by applying a small amount of force on elongated member 104 such that extensions 46 may be biased outward from an original position to provide sufficient clearance for a portion of elongated member 104 to seat in retaining member 40. In some embodiments, once elongated member 104 passes a certain point, extension 46 may be able to return to the original position and the inward force provided by extensions 46 retains elongated member 104 in retaining member 40.

In some embodiments, once elongated member 104 is properly in position, the surgeon may proceed to lock it in position. In some embodiments, assembly head 22 may include helical thread 24 as mentioned above for rotatable engagement of threads 52 on closure member 106. In some embodiments, closure member may have a generally cylindrical shape and possesses an “artillery” type thread 52 suitable for co-operating with the helical thread 24 of assembly head 22.

In some embodiments, when closure member 106 is threaded into assembly head 22, bottom surface 54 (depicted in FIG. 10) may contact the outside surface of elongated member 104. In some embodiments, a portion of closure member 106 may include tool portion 56. Tool portion 56 may allow detachable connection with a screwdriver, internal or external hexagonal socket, TORX style head, or any other standard or proprietary tool.

In some embodiments, the use of a thread of the “artillery” type may present the advantage of reducing radial force while tightening closure member 106. FIG. 12 depicts an exploded view of one embodiment of hook 10 for coupling with elongated member 104, in which artillery type threads 24 may engage threads 52 on closure member 106. In some embodiments, side walls 28, 28′ may no longer be subjected to a radial force tending to splay them apart from each other. In some embodiments, by using this type of helical threads, there may not be a need to use an external hooping ring for holding walls 28, 28′ at the desired spacing.

In some embodiments, even if there is any residual force tending to space walls 28, 28′ apart, retaining member 40 may continue to hold elongated member 104, because retaining member 40 and assembly head 22 may be mechanically decoupled, i.e., the mechanical forces to which assembly head 22 is subjected are not transmitted to retaining member 40.

In some embodiments, when closure member 106 couples hook 10 to elongated member 104 in a locked position (See, e.g., FIGS. 11 and 13), closure member 106 may be fully received inside assembly head 22 so the outside profile of hook 10 may be smooth. In other words, in some embodiments, hook 10 does not present any tapping, bulges, grooves, or roughness that might damage the surrounding tissue.

Similarly, in some embodiments, there are no additional parts (e.g., hooping rings) on the outside surface of assembly head 22 that might present sharp edges that could damage surrounding tissue.

In some embodiments, hook 10 may present smooth surfaces of other shapes, of the “smooth profile” type, that may be inserted reliably in the human body without any risk of provoking internal lesions. In some embodiments, hook 10 may be implanted in a portion of a vertebra. In some embodiments, hook 10 may be implanted in a spinous process. In some embodiments, hook 10 may be implanted in a transverse process. In some embodiments, hook 10 may be implanted in a pedicle.

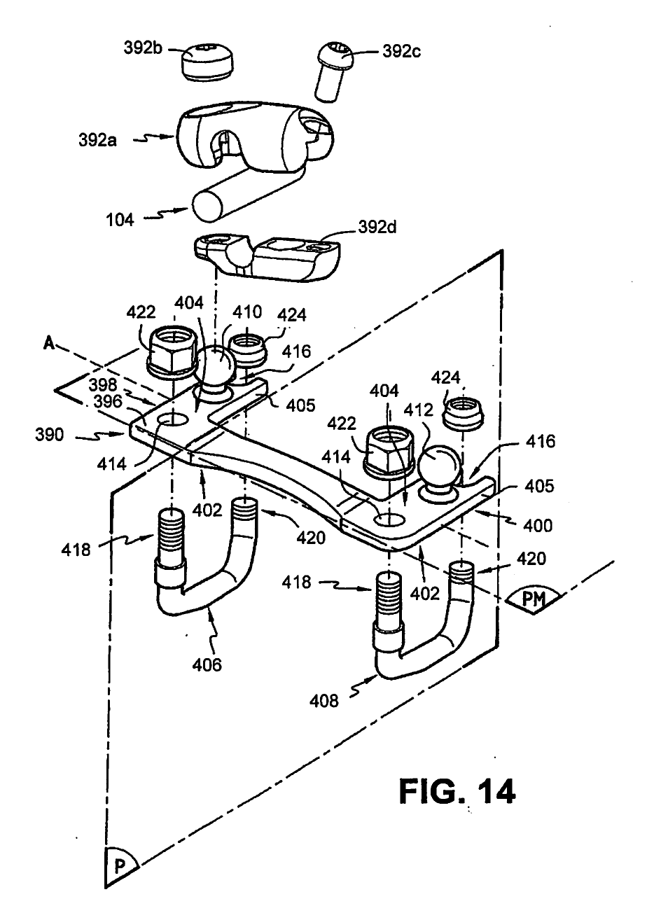

In some embodiments, a fastener such as a clamp may be useful for coupling elongated member 104 to a portion of a spine. FIG. 14 depicts an exploded view of one embodiment of clamp 390 suitable for receiving at least one receiver 392 enabling elongated member 104 to be coupled to a vertebra, with only a portion of elongated member 104 being shown in FIG. 14.

In some embodiments, clamp 390 may include transverse elongate part 396 of axis A defining a mean plane PM and presenting two opposite ends 398, 400. In some embodiments, two ends 398, 400 may be substantially mirror images of each other about a plane of symmetry P which intersects transverse elongate part 396 orthogonally. In some embodiments, each end 398 and 400 may have first main face 402 and second main face 404, together with end edge 405. In some embodiments, two connection elements 406 and 408 may be suitable for connection to ends 398 and 400. In some embodiments, ends 398 and 400 may have respective anchor means 410 and 412 of spherical shape projecting from second main faces 404.

In some embodiments, ends 398 and 400 may include first recesses 414 and second recesses 416 oriented away from first recesses 414. In some embodiments, first and second recesses 414 and 416 may be spaced apart from each other by a first distance. In some embodiments, first recesses 414 may be oriented on transverse part 396. In some embodiments, first and second recesses 414 and 416 may be positioned on first and second main faces 402 and 404. In some embodiments, an axis traveling through first and second recesses 414 and 416 may define a line that is substantially perpendicular to transverse part 396.

In some embodiments, connection elements 406 and 408 may form a substantially U-shaped part having free ends 418 and 420 spaced apart from each other by a distance corresponding to the distance between first and second recesses 414 and 416. In some embodiments, connection elements 406 and 408 may be male threaded for engaging female threaded members 422 and 424. In some embodiments, free ends 418 and 420 of connection elements 406 and 408 may be inserted into first and second recesses 414 and 416 with connection elements 406 and 408 coming respectively into register with the first main faces 402 of transverse part 396. In some embodiments, once free ends 418 and 420 have been inserted through first and second recesses 414 and 416 so as to project from second main face 404, threaded members 422 and 424 may be threadably engaged to retain connection elements 406 and 408 and to prevent transverse elongate part 396 from moving relative to the vertebra.

In some embodiments, the shape of second recess 416 may facilitate mounting connection elements 406 and 408 on transverse part 396. In some embodiments, the shape of second recesses 416 may make mounting quicker, due to second recess 416 opening out into edge face 405 and forming an oblong hole. In some embodiments, when transverse part 396 is in the installation position, threaded members 424 may be pre-mounted on free ends 420 of each connection member 406 and 408. In some embodiments, connection member 406 and 408 may be presented in a plane parallel to the plane P, with free end 420 directed towards second recess 416 so that connection member 406 and 408 may slope relative to the mean plane PM. When implanted in the body, connection member 406 and 408 may be parallel to the plane AR. In some embodiments, connection members 406 and 408 may engage transverse part 396 substantially parallel to the mean plane PM so that threaded member 424 may seat against second face 404. In some embodiments, this may enable connection members 406 and 408 to be pivoted about the point where threaded member 424 seats against second face 404, so that free end 418 which is initially situated facing first face 402 may engage recess 414. In some embodiments, free end 418 may be subsequently held in place by threaded member 422. In some embodiments, when connection member 406 or 408 pivots, it may be suitable for coupling with a process situated facing first face 402.

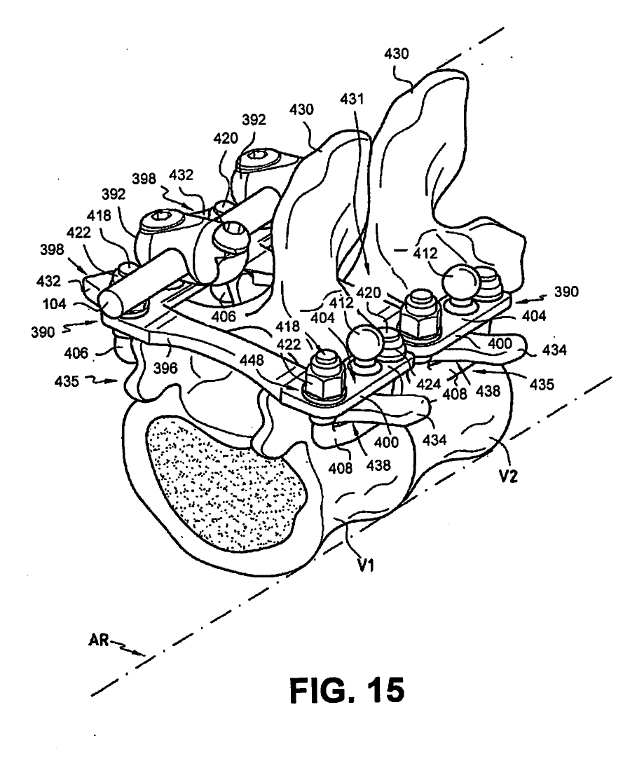

In some embodiments, connection part 406 and 408 may be deformable to conform against the outline of an object to provide better retention. In some embodiments, connection members 406 and 408 may be manufactured from polymers. In some embodiments, connection members 406 and 408 may be manufactured from polyethylene. In some embodiments, connection members 406 and 408 may be manufactured from biocompatible materials. In some embodiments, connection members 406 and 408 may be manufactured from single strands of material or multiple strands functioning as a single strand. In some embodiments, connection members 406 and 408 may be manufactured to conform to portions of the spine. In some embodiments, connection members 406 and 408 may be manufactured to conform to portion(s) of transverse processes 438 as depicted in FIG. 15.

In some embodiments, ends 418 and 420 of connection part 406 and 408 may include connection means and adjustment means that are suitable for the material. In some embodiments, ends 418 and 420 of connection part 406 and 408 may be crimped in a rigid material element suitable for being threaded or for constituting a shoulder (see e.g., shoulder 448 of FIG. 15). In some embodiments, ends 418 and 420 of connection part 406 and 408 may be overmolded on the free ends of the limbs. In other words, ends 418 and 420 may be manufactured as a part of connection part 406 and 408 or may be manufactured separately and joined to connection part 406 or 408.

In some embodiments, threaded members 422 and 424 may be irreversibly secured to ends 418 and 420 of connection parts 406 and 408 so that prolonged traction exerted on connection parts 406 and 408 by threaded members 422 and 424 does not cause them to separate.

In some embodiments, anchors 410 and 412 having substantially spherical heads may be situated on second main surfaces 404, between first and second recesses 414 and 416. In some embodiments, anchors 410 and 412 may be securely connected to transverse part 396 so that the mechanical forces exerted on anchors 410 and 412 can move transverse part 396 without leading to separation. In some embodiments, anchors 410 and 412 may be welded, machined, or otherwise securely connected to transverse part 396.

FIG. 15 depicts a perspective view of one embodiment of a spinal stabilization system on two adjacent vertebrae V1 and V2 forming a portion of the spine. Those skilled in the art will appreciate that this figure is presented for simplicity purposes and embodiments may be attached to non-adjacent vertebrae. Each vertebra may include spinous process 430 projecting from posterior wall and two transverse processes 432 and 434 projecting from sides 435. In some embodiments, one or more vertebrae V1 and V2 may be fitted with a clamp 390 as shown in FIG. 14. In some embodiments, two or more clamps 390 may be connected to each other by means of two receivers 392 which are coupled to elongated member 104. Receiver 392 may generally comprises bottom portion 392D for coupling to anchor 410 or 412 and having a channel therein to receive a portion of elongated member 104, top portion 392A having a channel therein for receiving a portion of elongated member 104, and one or more closure members 392B and 392C for connecting top portion 392A to bottom portion 392D to couple clamp 390 to elongated member 104. For illustrative purposes, only two vertebrae V1 and V2 are shown. Nevertheless, clamps 390 may be suitable for being mounted on several vertebrae. Similarly, only one elongated member 104 and only two receivers 392 are depicted in FIG. 15.

In some embodiments, transverse parts 396 may seat against posterior walls of vertebrae V1 and V2 substantially perpendicular to the axis AR of the spine. In some embodiments, transverse parts 396 may be held in this position by means of connection parts 408 and 406 whose free ends 418 and 420 may be held in first and second recesses 414 and 416 by means of threaded members 422 and 424 which press against second main faces 404. In some embodiments, tightening threaded members 422 and 424 about free ends 418 and 420 of connection parts 406 and 408, the bottoms of connection parts 406 and 408 may be pulled against the anterior walls 438 of transverse processes 432 and 434, and consequently ends 398 and 400 of transverse parts 396 may be pulled towards transverse processes 434 and 432. In some embodiments, transverse part 396 may be held on either side of the vertebra and against its posterior wall. In some embodiments, transverse part 396 may be completely prevented from moving relative to the vertebra. In some embodiments, threaded members 422 and 424 may be tightened to compress the bony wall a selected amount to further aid retention.

In some embodiments, once clamps 390 have been secured to vertebrae V1 and V2, receivers 392 coupled to elongated member 104 may be positioned and clamped on anchors 410 and 412 on both sides of the spine. In some embodiments, receivers 392 may be held in a fixed position relative to anchors 410 and 412 and elongated member 104 may be held in a fixed orientation relative to receivers 392 so that anchors 410 and 412 may be prevented from moving relative to one another. Consequently, vertebrae V1 and V2 may be held laterally in positions that are fixed relative to one another.

In some embodiments, connection elements 406 and 408 may be engaged solely around the transverse processes 432 and 434. The vertebrae shown in those figures do not carry any ribs. In some embodiments, clamps 390 may be coupled to vertebrae having ribs connected thereto. In some embodiments, connection parts 406 and 406 may have a length greater than connection parts 406 and 408 used for vertebrae that do not carry ribs. In some embodiments, longer connection parts 406 and 408 may be engaged around the ribs so that their bottoms bear against the anterior walls of the ribs and so that, as in the preceding embodiments, free ends 418 and 420 may pass through first and second recesses 414 and 416 of transverse part 396 in order to be secured thereto.

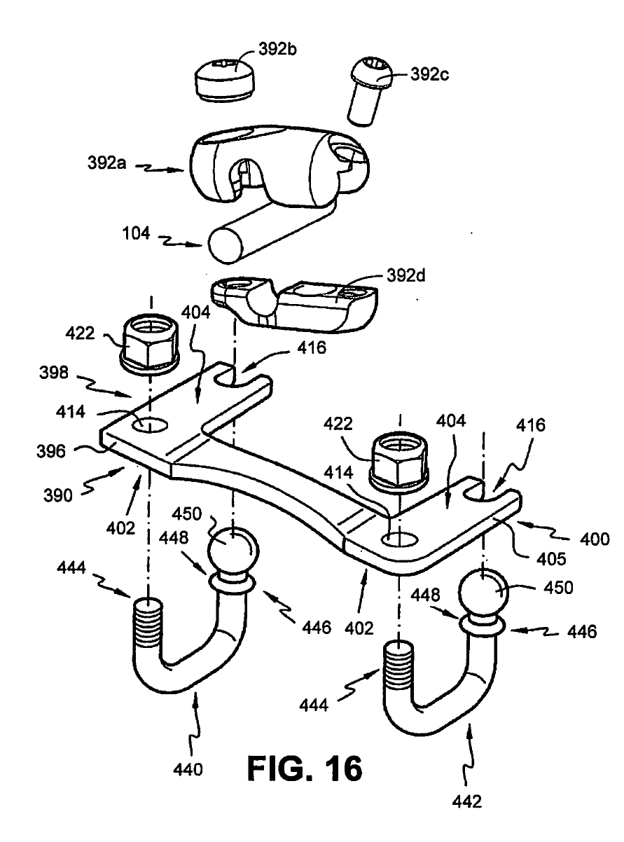

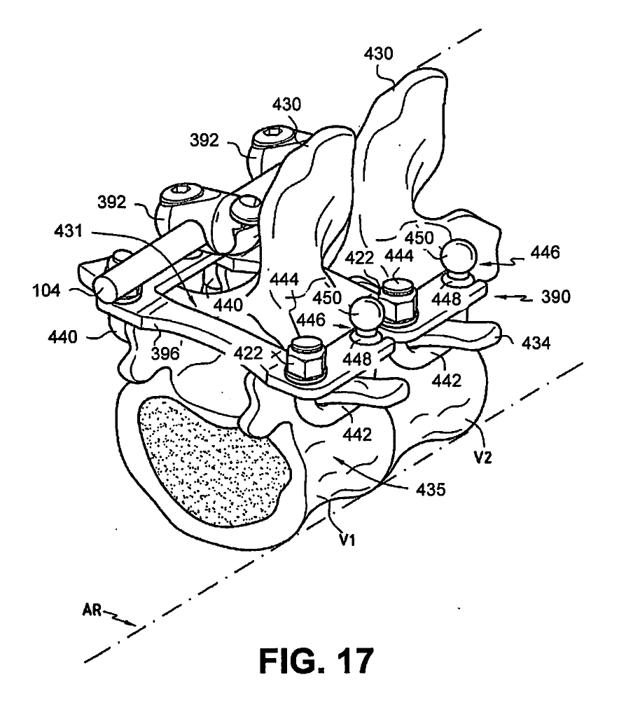

In some embodiments, receivers 392 may be mounted on the spherical heads 412 on both sides of the spine. FIG. 16 depicts an exploded perspective view of one embodiment of clamps 390. FIG. 17 depicts a perspective view of one embodiment of a spinal stabilization system with clamps 390 on vertebrae.

In some embodiments, connection parts 440 and 442 may have free end 444 and anchor 450. In some embodiments, free end 444 may be threaded to engage threads on threaded member 422. In some embodiments, free end 446 may include shoulder 448 surmounted by anchor 450.

In some embodiments, anchor 410 and 412 may be joined to transverse part 396 together with connection parts 406 and 408 with which it forms an integral part. In some embodiments, fitting of transverse part 396 to vertebra V1 or V2 may be simplified and more firmly anchor elongated member 104 to the vertebrae.

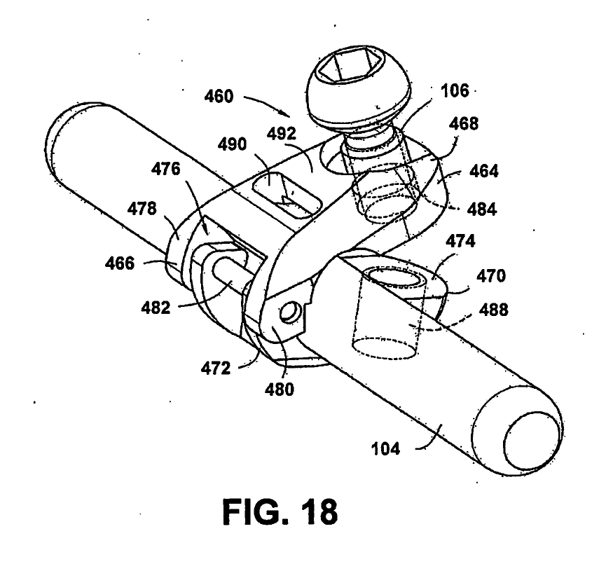

FIG. 18 depicts one embodiment of universal band 460 for coupling to elongated member 104, with universal band 460 open (i.e., in contact with but not coupled to elongated member 104). In some embodiments, universal band 460 includes connecting part 462 having two longitudinal members 464 and 470, of which first longitudinal member 464 extends between first end 466 and second end 468 and second longitudinal member 470 extends between first end 472 and second end 474. In some embodiments, longitudinal members 464 and 470 may be pivoted together at their first ends 466 and 472 for the purposes of coupling to elongated member 104. In some embodiments, first end 466 of longitudinal member 464 may have notch 476 with opposite edges 478 and 480 and between which first end 472 of longitudinal member 470 may be inserted. In some embodiments, pivot pin 482 may pass through first ends 466 and 472 and may be free to rotate in at least one of said ends 466 and/or 472. In some embodiments, second end 468 of first longitudinal member 464 may include bore 484 into which closure member 106 may be inserted. In some embodiments, second end 474 of second longitudinal member 470 may have helical thread 488 which may be aligned with bore 484 when longitudinal members 464 and 470 are disposed facing each other. In some embodiments, closure member 106 may be rotatably engaged with thread 488 in order for second ends 468 and 474 of longitudinal members 464 and 470 to face towards each other.

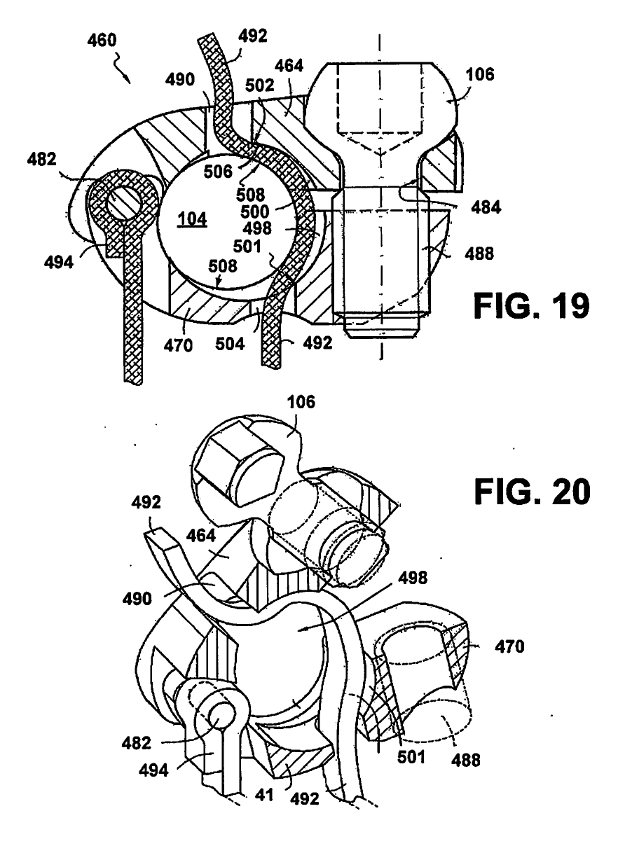

FIG. 19 depicts a cross-sectional view of one embodiment in which universal band 460 may include first orifice 490 through which ligature 492 may be passed. In some embodiments, passage 498, which may be symmetrical inside first longitudinal member 464, may be produced by a groove formed in each of the two facing faces of the middle parts of longitudinal members 464 and 470. In some embodiments, wall 508 may form an edge with cylindrical symmetry and corresponding with second portion 502 of the middle part of first longitudinal member 464 may form a substantially cylindrical space into which elongated member 104 may be inserted.

In some embodiments, universal band 460 may couple to elongated member 104 by capturing elongated member 104 between two longitudinal members 464 and 470 such that a portion of ligature 492 may be pressed against elongated member 104.

In some embodiments, pin 482 and elongated member 104 may be substantially perpendicular to the transverse process such that ligature 492 may need to be partly twisted in order to insert it into the passage 498 and between pin 482 and the point at which it contacts the transverse process.

In some embodiments, wall 508 of elongated member 104 may bear on top of elongated member 104 and one or more of walls 501, 502 and 506 may press flexible ligature 492 against elongated member 104. In some embodiments, closure member 106 may drive longitudinal members 464 and 470 forcibly against elongated member 104 and simultaneously against ligature 492, which may also be forcibly pressed against elongated member 104.

In some embodiments, passage 498 may have a width near orifice 504 greater than a section in the vicinity of the orifice 490. In some embodiments, the width of passage 498 may progressively decrease in the direction from orifice 504 to orifice 490. In some embodiments, flexible ligature 492 may be therefore progressively compressed around a portion of elongated member 104 with a pressure that increases in the direction from orifice 504 towards orifice 490.

In some embodiments, flexible ligature 492 may have a first end 494 that may be ligated around pin 482, and second end 496 that may be inserted into passage 498 between elongated member 104 and internal walls 500 and 501 of longitudinal members 464 and 470 and external wall of elongated member 104. In some embodiments, second longitudinal member 470 may include second orifice 504 through which ligature 492 may pass.

FIG. 20 depicts a cutaway view of one embodiment of universal band 460 through which ligature 492 may be passed. In some embodiments, universal band 460 may include first longitudinal member 464 and second longitudinal member 470, in which longitudinal members 464 and 470 may pivot about pin 482 that joins them, and ligature 492. In some embodiments, closure member 106 screwed into thread 488 of bore 484 may couple universal band 460 to elongated member 104 and fix in position a portion of flexible ligature 492.

In some embodiments, flexible ligature 492 may be an elongate flexible member capable of conforming to the contour of the parts that it must connect. In some embodiments, flexible ligature 492 may be a flexible strip of substantially constant width and thickness whose first end may be ligated to pin 482, the ligature surrounding the transverse process of the vertebra being inserted through connecting part 462. In some embodiments, ligature 492 may be manufactured from a polymer. In some embodiments, ligature 492 may be manufactured from polyester. In some embodiments, ligature 492 may be manufactured from DACRON polyester. In some embodiments, the thickness of the flexible ligature 492 may be substantially rectangular. In some embodiments, flexible ligature 492 may be made from a flexible material such as polyester that may be lightly crushed locally to immobilize it with a clamping effect.

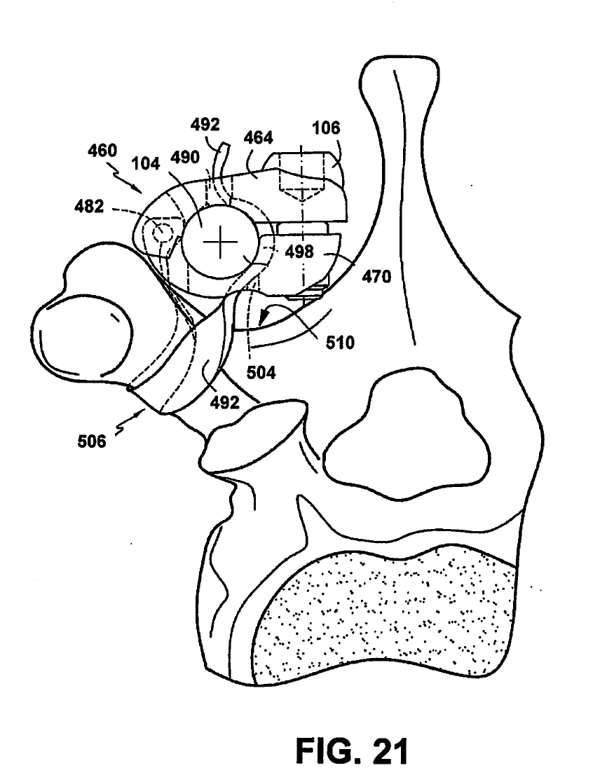

FIG. 21 depicts a perspective view of one embodiment of universal band 460 connected to a bone. In some embodiments, ligature 492 may be formed into loop 506 such that a portion of one or more bony tissues may be captured. In some embodiments, ligature 492 may capture a rib, spinous process, or pedicle.

In some embodiments, universal band 460 may be fixed in position against posterior wall 510 of the vertebra despite these partially twisted portions, the ligature 492 being forcibly tensioned by pulling second end 496. In some embodiments, universal band 460 may not be fixed against a portion of the spine.

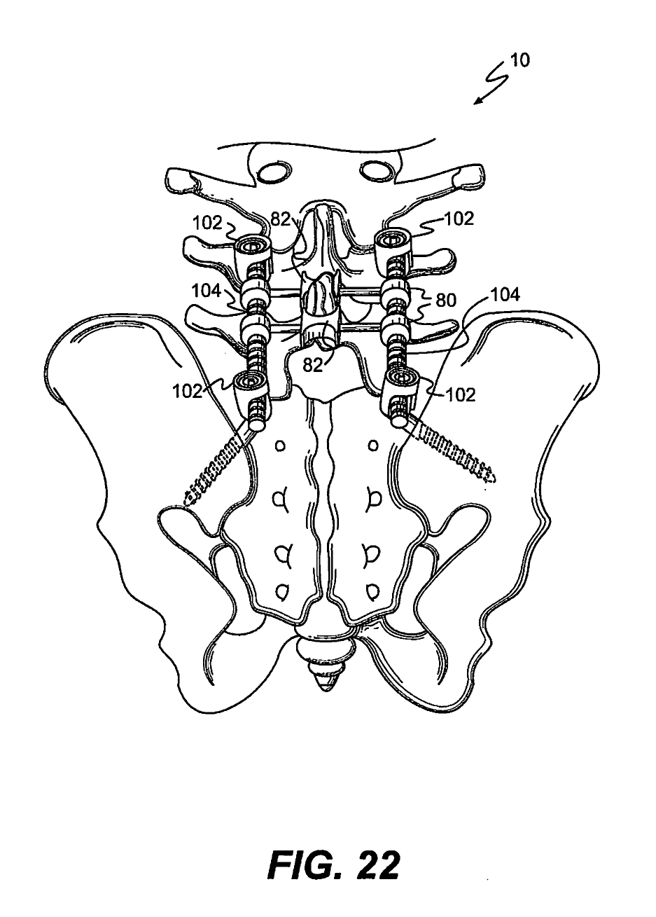

FIG. 22 depicts a view of a portion of a spine in which an exemplary stabilization system has been implanted. In this example, bone fasteners 108 have been implanted in pedicles on each vertebra and elongated members 104 have been coupled to bone fastener assemblies 102.

In one embodiment of the present disclosure, dynamic stabilization system 100 may include two elongated members 104 positioned on either side of the ridge formed by the spinous processes. Unlike prior art systems, however, bone fastener assemblies 102, hooks 10, bolts, clamps 390 or universal bands 460 may not be coupled to each vertebra. For example, in one embodiment depicted in FIG. 22, bone fastener assemblies 102 may be located only on a first vertebra such as lumbar vertebra L4 and a second vertebra such as the sacrum S. In other words, in some embodiments, there may be no bone fastener assemblies 102 located on a third vertebra such as L5. One result to this embodiment may be a reduced number of fasteners used in the stabilization system, which may be advantageous for reducing the time needed to surgically implant the system, reducing the amount of trauma to the body, and/or reducing the risk of damaging the spinal cord or other nerves, muscles or other tissues, or blood vessels either during or subsequent the surgery.

In some embodiments, dynamic stabilization system 100 may include one or more interspinous members 80. In some embodiments, interspinous members 80 may be coupled to one or both of elongated members 104. In some embodiments, body 82 of interspinous member 80 may be inserted or otherwise positioned between the spinous processes of adjacent vertebrae to stabilize movement between the spinous processes.

In some embodiments, the positioning of interspinous members 80 may provide stabilization and control movement between adjacent spinous processes. In some embodiments, the size, material, orientation, or any combination of body 82 may stabilize movement between adjacent spinous processes. In some embodiments, the positioning of lateral portions 88 on elongated members 104 may position body 82 of interspinous member 80 to stabilize movement between adjacent spinous processes. For example, in some embodiments, positioning lateral portions 88 closer to the lower of two spinous processes may effectively position body 82 of interspinous member 80 to provide more pressure (i.e., support) to the lower spinous process and less pressure (i.e., support) to the adjacent upper spinous process during flexion of the spine. In this example, the patient may have more support during one spinal movement than another (e.g., more flexion than extension) or one spinous process may be more supported or constrained than adjacent spinous processes.

In some embodiments, interspinous members 80 may have body 82 which may be deformable to stabilize movement. In some embodiments, body 82 may be deformed during contact with an inferior surface of a spinous process. In some embodiments, body 82 may return to its original state or a neutral state once pressure is relieved. In one embodiment, upper surface 84 and lower surface 85 may have any profile useful for contacting a spinous member. In one embodiment, body 82 may be manufactured from any material useful for providing dynamic support to a spine. In some embodiments, body 82 may be manufactured from PEEK (polyetheretherketone), UHMWPE (Ultra-High Molecular Weight Polyethylene), or any polymer or ceramic to provide dynamic support. In some embodiments, body 82 may be manufactured from a single material. In some embodiments, body 82 may be manufactured having constant properties such as density or elasticity. In some embodiments, body 82 may be manufactured having varying density. In some embodiments, body 82 may be manufactured from two or more materials. In some embodiments, body 82 may be manufactured with a core manufactured from one material or process and an outer region manufactured from a second material. In some embodiments, body 82 may be manufactured such that body 82 and/or interspinous member 80 may be implanted and then injected, treated, coated, shaped, filled, or otherwise modified to produce a final body 82 with selected geometry, elasticity, density, or other characteristic useful for controlling movement between adjacent vertebrae. In some embodiments, body 82 may be manufactured having a hollow core and injected with a fluid once implanted such that body 82 is positioned between adjacent vertebrae and upper surface 84 and lower surface 85 contact the spinous processes of adjacent vertebrae. In some embodiments, a set of interspinous members 80 having bodies 82 of different sizes, thicknesses, elasticity, density, core size, material, or other characteristics such that an appropriate interspinous member 80 may be selected. Advantageously, interspinous member 80 may be selected for stabilizing movement and then positioned between spinous processes to help stabilize the spine.

In some embodiments, interspinous member 80 may stabilize movement by limiting the maximum amount of travel between two adjacent vertebrae. In some embodiments, interspinous member 80 may stabilize movement by limiting the maximum amount of travel due the geometry of body 82. In some embodiments, interspinous member 80 may stabilize movement by limiting the maximum amount of travel due to positioning on elongated members 104.

In some embodiments, body 82 may stabilize movement by providing progressive resistance to cushion or dampen movement between adjacent vertebrae. In some embodiments, body 82 may have an associated spring constant. For example, in one embodiment first interspinous member 80 positioned between a first vertebra and a second vertebra may have body 82 with a stiff spring constant for greater rigidity, and second interspinous member 80 positioned between the second vertebra and a third vertebra may have a body 82 with a weak spring constant for greater flexibility. In this embodiment, the spine may have greater flexibility between the second and third vertebrae than between the first and second vertebrae. In some embodiments, body 82 may stabilize movement by providing a progressive resistance and may further have a maximum amount of travel. The selection of a spring constant and/or maximum amount of travel may depend or be determined based on patient comfort and health, surgical goals, or other criteria. In some embodiments, body 82 may stabilize movement by limiting the rate of movement between adjacent vertebrae. In other words, an interspinous member 80 positioned between two vertebrae may not limit the range of travel but may limit how fast the vertebrae may travel. In some embodiments, body 82 may stabilize movement by limiting the maximum rate at which adjacent vertebrae may move relative each other but may not limit the range of motion. In some embodiments, body 82 of interspinous member 80 may stabilize one or more directions of movement. In some embodiments, body 82 may have generally flat surfaces 84 and 85 that may not affect rotational movement. In some embodiments, body 82 may have concave surfaces 84 and 85 that may impede rotational movement. In some embodiments, body 82 may have asymmetric surfaces 84 and 85 that may bias movement toward one side.

In some embodiments, elongated members 104 may be coupled to one or more vertebrae. In some embodiments, an elongated member may be coupled to a vertebra using bone fastener assembly 102. In some embodiments, bone fastener assembly 102 may be implanted in a pedicle, a spinous process, or a transverse process. FIG. 22 depicts a perspective view in which two elongated members 104 may be coupled to a spine. In some embodiments, the upper ends of elongated members 104 may be coupled to bone fastener assemblies 102 that may be implanted in pedicles. In some embodiments, the lower portions of elongated members 104 may be coupled to bone fastener assemblies 102. However, there may be no bone fasteners, hooks, bolts, clamps, or universal bands coupled to the middle vertebra, and interspinous members 80 provide dynamic stabilization between the middle vertebra and the adjacent vertebrae.

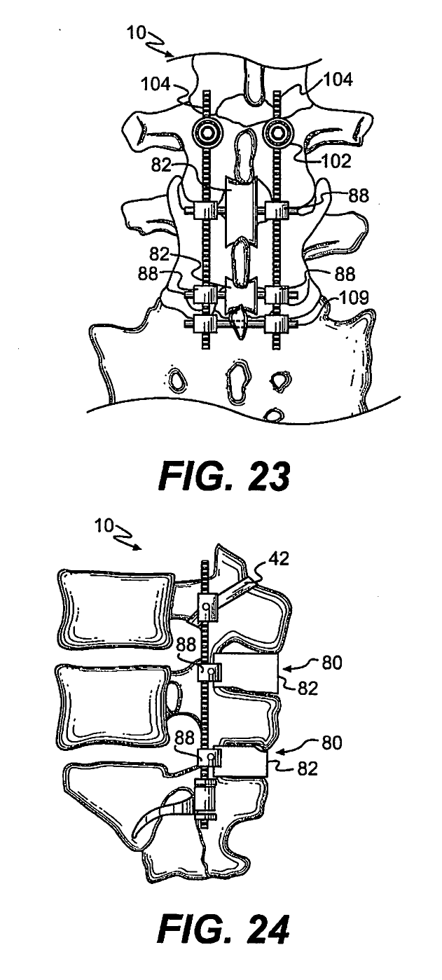

FIG. 23 depicts a posterior view of a spine in which upper portions of elongated members 104 may be coupled to bone fastener assemblies 102 coupled to pedicles in a vertebra. The lower portions of elongated members 104 may be coupled to either end of a bolt 109 passing through a portion of a spinous process. In some embodiments, there may be no bone fasteners, hooks, bolts, clamps, or universal bands coupled to the middle vertebra and interspinous members 80 may provide dynamic stabilization between the middle vertebra (e.g., L5) and the adjacent vertebrae (e.g., L4 and S1).

In some embodiments, interspinous member 80 may stabilize movement between elongated members 104. In some embodiments, lateral portions 88 and body 82 may form a cross-link mechanism for stabilizing movement between elongated members 104. In some embodiments, lateral portions 88 and body 82 may include a cross-link mechanism useful for stabilizing movement between elongated members 104. As used herein, the term cross-link may refer to a device that connects to both elongated members 104 and limits or eliminates movement of one elongated member 104 relative to the other. In some embodiments, a cross-link may limit or eliminate motion in one or more planes, or about one or more axes of rotation.

In some embodiments, one or more ends of elongated members 104 may be coupled to a vertebra using hook 10. In some embodiments, elongated members 104 may be coupled to one or more universal bands 460 affixed to a portion of a spine. FIG. 24 depicts a sagittal view of one embodiment in which the upper end of elongated member 104 may be coupled to universal band 460 affixed to a vertebra with flexible ligature 492 and the lower end may be coupled to hook 10 such that elongated member 104 may be coupled to the vertebrae and positioned parallel with the spinous processes. In some embodiments, there may be no bone fasteners 102, hooks 10, bolts 109, clamps 390, or universal bands 460 coupled to the middle vertebra and interspinous members 80 may provide dynamic stabilization between the middle vertebra and the adjacent vertebrae. In some embodiments, elongated members 104 may have one or more engagement features to facilitate alignment, positioning, or securing of interspinous members 80 to elongated members 104. In some embodiments, elongated member 104 may have a series of alignment marks. In some embodiments, elongated member 104 may have a series of notches 11. In some embodiments, notches 11 may be allow one way movement of interspinous members 80 on elongated members 104.

In some embodiments, elongated members 104 may be coupled to a portion of the spine using clamps (e.g., clamp 390) or similar devices. In some embodiments, elongated members 104 may be coupled directly to a portion of a spinous process. In some embodiments, elongated members 104 may be coupled together. In some embodiments, elongated members 104 coupled together may form a slot to accommodate the spinous processes. In some embodiments, the slot accommodates the spinous processes by providing sufficient clearance for the spinous processes to have some lateral movement. In some embodiments, the slot accommodates the spinous processes by capturing the spinous processes to inhibit lateral movement.

In some embodiments, elongated members 104 may be coupled to one or more universal bands 460 coupled to a spinous process. In some embodiments, elongated members 104 may be coupled together using universal band 460 to accommodate one or more spinous processes by dynamic stabilization system 100. For example, in some embodiments, elongated members 104 may be banded to form a slot to accommodate one or more spinous processes. In some embodiments, the slot accommodates the spinous processes by providing sufficient clearance for the spinous processes to have some lateral movement. In some embodiments, the slot accommodates the spinous processes by capturing the spinous processes to inhibit lateral movement.

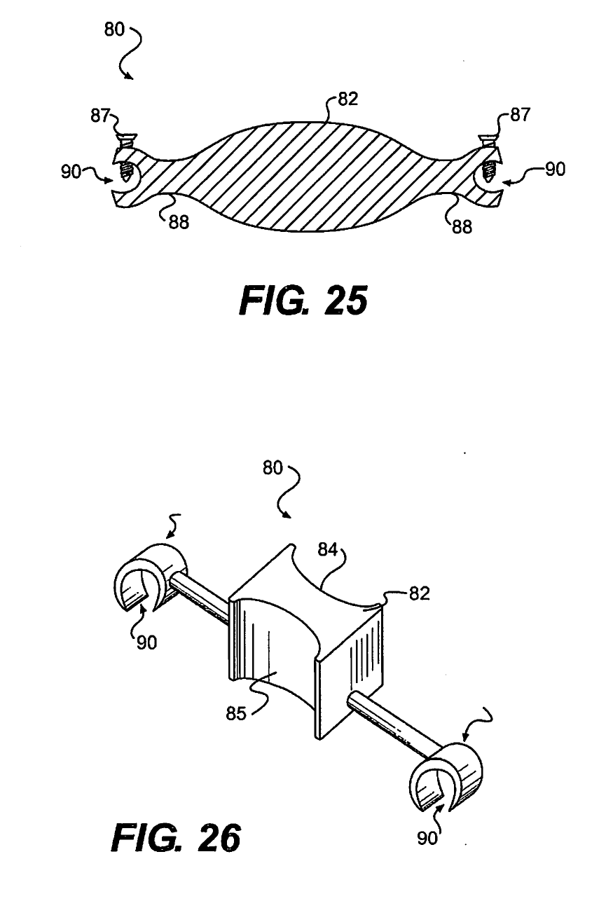

Embodiments of interspinous members 80 disclosed herein may be particularly useful in minimally invasive surgery (MIS) procedures or in non-MIS procedures, as desired, and as persons of ordinary skill in the art who have the benefit of the description of the disclosure understand. FIGS. 25 and 26 depict views of embodiments of interspinous members 80 having body 82 that may be positioned between adjacent spinous processes to stabilize movement between the spinous processes and thus the vertebrae. FIG. 25 depicts a cross-sectional view of one embodiment of interspinous member 80, having body 82 for allowing selected movement between the spinous processes of adjacent vertebrae and lateral portions 88 for engagement with elongated members 104. In some embodiments, interspinous member 80 may have a unibody construction such that a cross-link mechanism may be integrated with interspinous member 80 via body 82 for stabilizing movement between elongated members 104.

In some embodiments, interspinous member 80 may include connection members 87 for maintaining the connection between elongated members 104 and interspinous member 80 to form a cross-link mechanism for stabilizing movement between elongated members 104. One advantage to connection members 87 is that interspinous member 80 may engage elongated member 104 and be positioned such as by sliding lateral portions 88 along elongated members 104 before locking interspinous member 80 in position.

FIG. 26 depicts a perspective view of one embodiment of interspinous member 80 in which body 82 may be manufactured separately from lateral portions 88 and then connected. In some embodiments, interspinous members 80 may have upper surface 84 for contact with an inferior surface of a spinous process. In one embodiment, upper surface 84 may have an essentially flat portion in contact with an inferior surface of a spinous process. In one embodiment, upper surface 84 may have a concave portion in contact with an inferior surface of a spinous process. In one embodiment, upper surface 84 may have a convex portion for contact with an inferior surface of a spinous process. In one embodiment, upper surface 84 may have a layered, coated, or textured portion for contact with an inferior surface of a spinous process.

In some embodiments, interspinous members 80 may have lower surface 85 for contact with a superior surface of a spinous process. In one embodiment, lower surface 85 may have an essentially flat portion in contact with a superior surface of a spinous process. In one embodiment, lower surface 85 may have a concave portion in contact with a superior surface of a spinous process. In one embodiment, lower surface 85 may have a convex portion in contact with a superior surface of a spinous process. In one embodiment, lower surface 85 may have a layered, coated, or textured portion for contact with a superior surface of a spinous process.

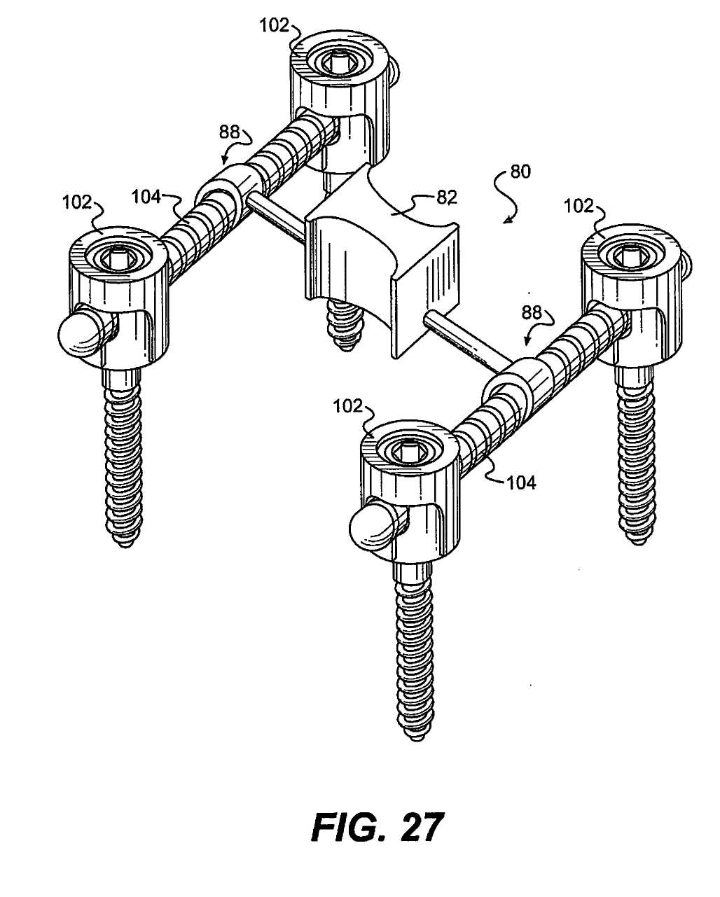

In some embodiments, interspinous members 80 may have two lateral portions 88 coupled to interspinous body 82, with each lateral portion 88 for engaging one elongated member 104. In some embodiments, lateral portions 88 include receiver portions 90 for receiving elongated members 104. In some embodiments, receiving portion 90 may be an open passage. In one embodiment, receiving portion 90 may be oriented facing lateral to the spine. In some embodiments, receiving portion 90 may be oriented posterior. In one embodiment, receiving portion 90 may be oriented anterior. In some embodiments, receiving portion 90 may connect to a portion of elongated member 104 with a compression fit. In other words, in some embodiments, receiving portion 90 may be temporarily expanded such that elongated member 104 may be positioned inside, then allowed to return to an original or neutral state such that elongated member 104 remains in receiving portion 90. In the embodiment depicted in FIG. 27, lateral portions 88 may couple with elongated members such that a set screw (e.g., connection member 87 in FIG. 25) may not be necessary.

In some embodiments, receiving portion 90 may be a closed passage (not shown) which may require interspinous members 80 to couple with elongated members 104 prior to coupling elongated members 104 to bone fastener assemblies 102, hooks 10, bolts 109, clamps 390, or universal bands 460. In some embodiments, interspinous members 80 may couple to elongated members 104 after elongated member 104 has coupled with one or more bone fastener assemblies 102, hooks 10, bolts 109, clamps 390, or universal bands 460.

FIG. 27 depicts a perspective view of spinal stabilization system 100 having four bone fastener assemblies 102 for coupling to vertebrae, two elongated members 104 for coupling bone fastener assemblies 102, and interspinous member 80 for coupling with elongated members 104.

In some embodiments, elongated members 104 may have engagement features 11. In some embodiments, engagement features 11 may be engaged by lateral portions 88. In some embodiments, engagement features 11 may appear on fluoroscopic images to enable a surgeon to position lateral portion 88 at a desired level.

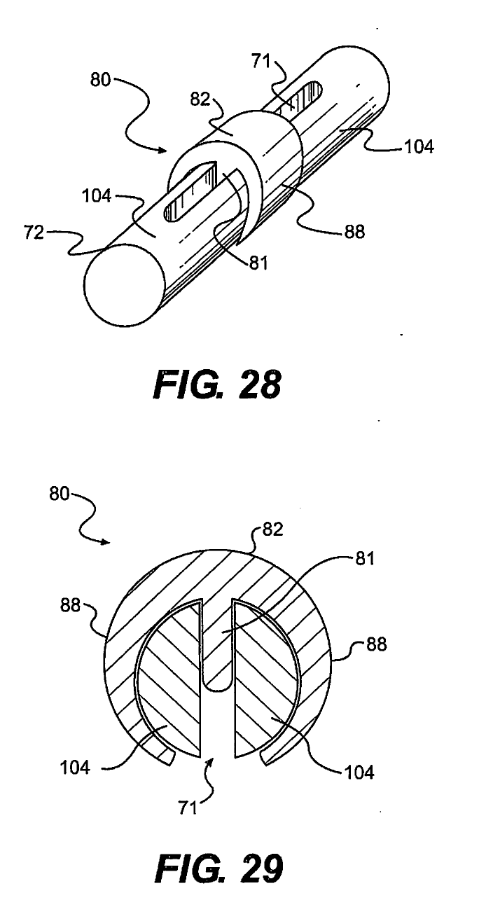

FIG. 28 depicts one embodiment in which elongated members 104 may be connected at either or both ends to form a unitary piece having a slot for accommodating two spinous processes. In some embodiments, elongated members 104 may be manufactured from a single rod (e.g., rod 72) such that elongated members 104 may refer to portions of rod 72. In one embodiment, elongated members 104 may be formed by machining slot 71 in a single rod 72. In some embodiments, connecting elongated members 104 at both ends forms a unitary piece having a slot 71 along a portion thereof. In some embodiments, elongated members 104 may be manufactured and then connected. In one embodiment, elongated members 104 may have complementary features on either or both ends to enable connection.

FIG. 29 depicts a cross-sectional view taken along line B-B′ of FIG. 28 of one embodiment of interspinous member 80 having curved lateral portions 88. In some embodiments, interspinous member 80 may have lateral portions 88 curved for receiving elongated members 104 such that receiving portion 90 may not be necessary.

In some embodiments, interspinous member 80 may include alignment feature 81 for positioning on elongated members 104 such that interspinous member 80 remains in position subsequent surgery. In some embodiments, interspinous member 80 may be aligned with elongated members 104 such that alignment feature 81 seats in slot 71. In some embodiments, once alignment feature 81 has been seated in slot 71, alignment feature 81 may prevent interspinous member 80 from rotating about or disconnecting from elongated members 104. Variations of lateral portions 88 and alignment feature 81 may be possible without departing from the scope of the disclosure.

In some embodiments, a spine stabilization system may include extensions, openings, tabs, recesses, or other features (not shown) to enable bone fasteners, hooks, bolts, clamps, or universal bands to couple elongated members 104 to one or more vertebrae. In some embodiments, once elongated members 104 are coupled to vertebrae, one embodiment of interspinous member 80 may be inserted into the patient. In some embodiments, prior to insertion of interspinous members 80, the tissue wedge or targeting needle may be used to wand between bone fasteners 102 to ensure a clean plane between the bone fasteners 102. An end of interspinous member 80 may be inserted. Inserting interspinous members 80 at an angle or substantially longitudinally allows the length of the incision and/or the area of the tissue plane to remain advantageously small. In some embodiments, interspinous member 80 may be positioned between first and second spinous processes.

In some embodiments of interspinous member 80 having curved lateral portions 88 to receive elongated members 104, lateral portions 88 may be expanded to receive elongated members 104. Once lateral portions 88 have received at least a portion of elongated members 104, lateral portions 88 may be released or otherwise returned to an original orientation to engage elongated members 104. In some embodiments, lateral portions 88 of interspinous member 80 may be curved, angled, bent, or otherwise shaped to receive at least a portion of elongated member 104. In some embodiments, lateral portions 88 may be shaped to extend around elongated members 104 (i.e., form a closed passage).

In some embodiments, lateral portions 88 may be positioned relative to one or both adjacent spinous processes, one or both transverse processes, or some other anatomical landmark. In some embodiments, engagement features 11 may enable lateral portions 88 to securely engage elongated members 104.

Lateral portions 88 of interspinous member 80 may be positioned on portions of elongated members 104. Engagement features on interspinous member 80 may couple to engagement features 11 on elongated members 104. Engagement features may include, but are not limited to, notches, grooves, threads, holes, openings, indentations, alignment marks, pawls, protrusions, and the like.

In the foregoing specification, the disclosure has been described with reference to specific embodiments. However, one of ordinary skill in the art appreciates that various modifications and changes can be made without departing from the scope of the disclosure as set forth in the claims below. Accordingly, the specification and figures are to be regarded in an illustrative rather than a restrictive sense, and all such modifications are intended to be included within the scope of disclosure.

The foregoing specification and accompanying figures are for the purpose of teaching those skilled in the art the manner of carrying out the disclosure and should be regarded in an illustrative rather than a restrictive sense. As one skilled in the art can appreciate, embodiments disclosed herein can be modified or otherwise implemented in many ways without departing from the spirit and scope of the disclosure and all such modifications and implementations are intended to be included within the scope of the disclosure as set forth in the claims below.

What is claimed is:

1. An interspinous member for stabilizing movement between adjacent vertebrae, comprising:
a body for stabilizing movement between a first spinous process and a second spinous process, having a first surface for contact with an inferior portion of the first spinous process and a second surface for simultaneous contact with a superior portion of the second spinous process; and
two lateral portions extending from the body for engaging two elongated members positioned lateral to the first spinous process and the second spinous process to stabilize movement between the elongated members.

2. The interspinous member of claim 1, wherein the upper surface has a curved portion for contact with a portion of the inferior portion of a spinous process.

3. The interspinous member of claim 1, wherein the lower surface has a curved portion for contact with a portion of the superior portion of a spinous process.

4. The interspinous member of claim 1, wherein each lateral portion is curved to receive a portion of an elongated member such that the lateral portion engages the elongated member.

5. The interspinous member of claim 1, wherein each lateral portion comprises a receiving portion for receiving a portion of an elongated member.

6. A system for stabilizing movement between two or more vertebrae, comprising:
two elongated members for positioning lateral to two spinous processes of the vertebrae;
two fasteners for coupling each elongated member to the vertebrae; and
an interspinous member for coupling to the two elongated members for positioning between the spinal processes of adjacent spinous processes, wherein each interspinous member comprises:
a body for stabilizing movement between a first spinous process and a second spinous process, having a first surface for contact with an inferior portion of the first spinous process and a second surface for simultaneous contact with a superior portion of the second spinous process; and
two lateral portions extending from the body for engaging the two elongated members positioned lateral to the first spinous process and the second spinous process to stabilize movement between the elongated members,
wherein positioning the lateral portions on the elongated members positions the body to stabilize movement between adjacent spinous processes.

7. The dynamic spinous process stabilization system of claim 6, wherein a fastener comprises a bone fastener assembly.

8. The dynamic spinous process stabilization system of claim 6, wherein a fastener comprises a hook.

9. The dynamic spinous process stabilization system of claim 6, wherein a fastener comprises a bolt for passage through a portion of a spinous process.

10. The dynamic spinous process stabilization system of claim 6, wherein a fastener comprises a clamp.

11. The dynamic spinous process stabilization system of claim 6, wherein a fastener comprises a universal band.

12. The dynamic spinous process stabilization system of claim 6, wherein the two elongated members are connected at both ends and comprise a slot along a portion thereof to accommodate two spinous processes.

13. The dynamic spinous process stabilization system of claim 6, wherein a slot formed along a portion of a single rod divides the rod into two elongated members for positioning lateral to two spinous processes.

14. The dynamic spinous process stabilization system of claim 6, wherein the two elongated members comprise an engagement feature, wherein one or more interspinous members engage the elongated members at one of the engagement features.

15. A method for stabilizing a portion of a spine, comprising the steps of:
coupling a first elongated member to a first side of a first vertebra;
coupling the first elongated member to the first side of a second vertebra, wherein the first elongated member spans between the first and second vertebrae;
coupling a second elongated member to a second side of the first vertebra;
coupling the second elongated member to the second side of the second vertebra, wherein the second elongated member spans between the first and second vertebrae;
positioning a body of an interspinous member between adjacent spinous processes such that a first surface of the body contacts an inferior portion of a first spinous process and a second surface contacts a superior portion of a second spinous process, wherein the body stabilizes movement between the adjacent spinous processes;
engaging two lateral portions of the interspinous member to a portion of the two elongated members, wherein each lateral portion for receiving a portion of an elongated member,
wherein the positioning of the lateral portions on the elongated members positions the body to stabilize movement between adjacent spinous processes.

16. The method of claim 15, comprising the step of:
slidably positioning a lateral portion along the first or second elongated member.

17. The method of claim 15, comprising the step of:
engaging the interspinous member with an engagement feature on an elongated member.

18. The method of claim 15, comprising the step of:
coupling an elongated member to a bone fastener assembly implantable in bony tissue.

19. The method of claim 15, comprising the step of:
coupling an elongated member to a hook implantable in bony tissue.

20. The method of claim 15, comprising the step of:
coupling an elongated member to a portion of a bolt assembly for penetrating through a portion of a spinous process.

21. The method of claim 15, comprising the step of:
coupling one of the elongated members to the other elongated member.

22. The method of claim 15, comprising:
coupling one of the elongated members to the other elongated member with a universal band.
